(19) United States
(12) Patent Application Publication
(10) Pub. No.: US 2006/0264917 A1
(43) Pub. Date: Nov. 23, 2006

(54) Scleral lenses for custom optic evaluation and visual performance improvement

(75) Inventors: Kuang-mon Ashley Tuan, Castro Valley, CA (US); Mark Arnoldussen, San Carlos, CA (US); Gunagming Dai, Fremont, CA (US); Kingman Yee, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: VISX, Incorporated, Santa Clara, CA (US)

(21) Appl. No.: 11/134,861

(22) Filed: May 20, 2005

(51) Int. Cl. A61B 18/18

(52) U.S. Cl. 606/5

(57) ABSTRACT

Methods and systems can correct aberrations and/or verify various procedures used to correct aberrations in the eye. One embodiment provides a method for verifying vision correction for a patient's eye comprising measuring irregular aberrations of the eye and determining a proposed refractive correction for treatment of the eye. A central portion of a verification lens is configured so that the central portion corresponds with the proposed correction. The verification lens is then registered with the eye by positioning a peripheral portion of the verification lens upon the sclera so that the central portion is optically aligned with the aberrations. Then a determination is made whether a corrected vision of the eye with the verification lens is acceptable so as to verify the proposed correction.

### BACKGROUND OF THE INVENTION

### Field of the Invention

Embodiments of the present invention are generally related to vision correction systems. In one embodiment, the invention provides systems and methods for verifying a laser refractive procedure, ideally by ablating a customized corrective scleral contact lens before imposing a corresponding refractive correction in the corneal tissues.

Known laser eye procedures generally employ an ultraviolet or infrared laser to remove a microscopic layer of stromal tissue from the cornea of the eye to alter the refractive characteristics of the eye. The laser removes a selected shape of the corneal tissue, often to correct refractive errors of the eye. Ultraviolet laser ablation results in photodecomposition of the corneal tissue, but generally does not cause thermal damage to adjacent and underlying tissues of the eye. The irradiated molecules are broken into smaller volatile fragments photochemically, directly breaking the intermolecular bonds.

Laser ablation procedures can remove the targeted stroma of the cornea to change the cornea's contour for varying purposes, such as for correcting myopia, hyperopia, astigmatism, and the like. Control over the distribution of ablation energy across the cornea may be provided by a variety of systems and methods, including the use of ablatable masks, fixed and moveable apertures, controlled scanning systems, eye movement tracking mechanisms, and the like. In known systems, the laser beam often comprises a series of discrete pulses of laser light energy, with the total shape and amount of tissue removed being determined by the shape, size, location, and/or number of a pattern of laser energy pulses impinging on the cornea. A variety of algorithms may be used to calculate the pattern of laser pulses used to reshape the cornea so as to correct a refractive error of the eye. Known systems make use of a variety of forms of lasers and/or laser energy to effect the correction, including infrared lasers, ultraviolet lasers, femtosecond lasers, wavelength multiplied solid-state lasers, and the like. Alternative vision correction techniques make use of radial incisions in the cornea, intraocular lenses, removable corneal support structures, thermal shaping, and the like.

Known corneal correction treatment methods have generally been successful in correcting standard vision errors, such as myopia, hyperopia, astigmatism and the like. However, as with all successes, still further improvements would be desirable. Toward that end, wavefront measurement systems are now available to measure the refractive characteristics of a particular patient's eye. By customizing an ablation pattern based on wavefront measurements, it may be possible to correct minor regular and/or irregular refractive errors so as to reliably and repeatably provide visual acuities of 20/20 or better. Unfortunately, these measurement systems are not immune from measurement error. Similarly, the calculation of the ablation profile, the transfer of information from the measurement system to the ablation system, and the operation of the ablation system all provide opportunities for the introduction of errors, so that the actual long term visual acuities provided by real-world wavefront-based correction systems may not be as good as might be theoretically possible.

In light of the above, it would be desirable to provide improved vision correction systems and methods.

### SUMMARY OF THE INVENTION

Various embodiments of the invention provide methods and systems for verifying procedures used to correct aberrations in the eye resulting in vision defects such as myopia, etc. Particular embodiments are useful for pre-operatively verifying the effectiveness of laser eye surgical procedures such as photorefractive keratectomy (PRK), phototherapeutic keratectomy (PTK), laser in situ keratomileusis (LASIK), and the like.

In a first aspect, the invention provides a method for verifying vision correction for an eye of a patient. The method comprises measuring irregular aberrations of the eye. A determination is made for a proposed refractive correction for treatment of the eye. The determination can be based on the measured aberrations or other optical evaluation of the eye. A central portion of a verification lens is configured to correspond with the proposed correction. A peripheral portion of the verification lens is positioned upon the sclera of the eye so that the central portion is optically aligned with the aberrations. This can be accomplished by registering the verification lens with the eye. Then a determination is made whether the corrected vision of the eye with the verification lens is acceptable. This determination is used to verify the proposed correction. The determination can include an evaluation of one or more of visual acuity, accommodation and contrast sensitivity as well the reading of an eye chart. The determination can be made after the verification lens has been worn for a period of hours, a day or even multiple days. Also, several determinations can be made over a desired period and the results compared (e.g., by quantitative or qualitative means). Various embodiments of the method can be used to evaluate a number of eye treatments including laser refractive treatments and the like. Also, in many embodiments, the irregular aberrations or other optical errors of a patient's eye can be measured with a wavefront sensor which, in specific embodiments, can be configured to measure refractive error. Measurements from the wavefront sensor can be used to produce a wavefront shape which can be used to configure the verification lens to correspond to the proposed correction. For example, in one embodiment, the wavefront shape is used to generate an ablation pattern (described below) for fabrication of the verification contact lens or a corrective contact lens worn by the patient on a long term basis.

In various embodiments, a treatment portion of the lens can have an aspheric shape configured to correspond to a proposed correction to treat various conditions of the eye such as refractive errors, higher order aberrations and presbyopia. Typically, the treatment portion comprises a central portion of the lens, but can comprise a non-central portion or even the entire lens.

In various embodiments, the peripheral portion of the lens can be configured to stabilize or otherwise reduce movement of the verification lens. For example, in one embodiment, the peripheral portion is used to stabilize the verification lens during determination of the corrected vision. This can be accomplished by configuring the peripheral portion to have a surface contour corresponding to a surface contour of the sclera so that the peripheral contour stabilizes the verification lens on the eye. The peripheral portion can also be used to reduce movement of the verification lens such as that which may result from blinking, eye movement (e.g., nystagmus) or head movement or a combination thereof. Also, the peripheral portion can be used to facilitate registration by supporting a substantial portion of the verification lens on the eye.

In various embodiments, the verification pattern can be an ablation pattern. The ablation pattern can be generated based on a proposed refractive correction treatment of the eye. The ablation pattern for the verification lens can be calculated from the measured irregular aberration of the eye, and from characteristics of the lens material, such as a refractive index of the lens material, a rate of ablation of the lens material, and/or ablation properties of the lens material (e.g., the propensity of the lens material to differ in ablation depth across a uniform ablation energy beam, such as any “central island” properties of the lens material). A corneal tissue of the eye may also be ablated according to an ablation pattern, and the ablation pattern may similarly be calculated based on the measured optical error of the eye and on the corneal tissue characteristics, such as a refractive index of the corneal tissue, a rate of ablation of the corneal tissue, and/or a shape of ablation of the corneal tissue. In many embodiments, the wavefront shape can be used to generate the ablation pattern to produce a corrective scleral lens which can be worn by the patient on a long term basis similar to conventional correct contacts lenses known in the art. (e.g., daily wear, extended wear, etc.)

In another aspect, the invention provides a method for forming a lens used to verify vision correction treatment for an eye of a patient or a corrective lens configured to be worn by the patient on a long term basis. The method includes measuring irregular aberrations of the eye and then determining a proposed refractive correction for treatment of the eye. An ablation pattern is then calculated based on the refraction correction, wherein the lens ablation pattern corresponds to an intended eye ablation pattern. A lens work piece is provided which has a central portion and a peripheral portion. The peripheral portion is configured to be positioned on the sclera of the eye. The lens workpiece is then ablated using an ablation system such that the ablation pattern is imposed on the central portion. In an exemplary method, the ablation system can be a laser ablation system but other lens shaping equipment and processes such as lathing or milling are equally applicable. Optionally, the lens workpiece can be a plano lens.

In still another aspect, the invention provides a system for correcting and/or verifying correction of irregular aberrations of an eye of a patient. The system includes a sensor for measuring the irregular aberrations of the eye and a processor for generating a verification pattern of laser energy corresponding to a refractive procedure plan of the eye. The procedure plan is intended to correct the measured irregular aberrations. The verification pattern can be an ablation pattern corresponding to an intended ablation pattern for treatment of the eye according to the procedure plan. The system also includes a lens workpiece and a laser system for directing laser energy onto the lens workpiece according to the verification pattern such that optical properties of the eye, as corrected by the verification lens, can verify the procedure plan. The workpiece has a central portion and a peripheral portion with the peripheral portion being configured to be positioned on the sclera. In various embodiments, the peripheral portion can be configured to optically align the central portion with aberrations on the eye, support a substantial portion of lens on the eye as well as stabilize the lens on the eye.

In other aspects, the invention also provides related systems for verifying and/or correcting various optical errors of an eye.

### BRIEF DESCRIPTION OF THE DRAWINGS

FIG. 1 is an overview of a laser ablation system.

FIG. 2 is a flow chart schematically illustrating an exemplary ablation verification and optical error correction method.

FIG. 3A illustrates a corneal lens positioned over the cornea of the eye.

FIG. 3B illustrates an embodiment of a scleral lens positioned over the cornea and sclera of the eye.

FIG. 3C illustrates an embodiment of a scleral lens having an aspheric shape.

FIG. 3D illustrates an embodiment of a scleral lens having a peripheral portion contour configured to stabilize the lens on the eye.

FIG. 3E illustrates an embodiment of a scleral lens having a peripheral portion surface area configured to stabilize the lens on the eye.

FIGS. 4A-C illustrate materials and lens assemblies that can be used to fabricate a verification contact lens, FIG. 4A illustrates a lens blank, FIG. 4B schematically illustrates a plano lens, and FIG. 4C illustrates a lens fitting set.

FIG. 5 is a schematic diagram illustrating an exemplary method for fabricating a verification contact lens.

FIG. 6 is a schematic diagram illustrating an exemplary method for fitting a verification contact lens.

FIGS. 7A-7B illustrate embodiments of a verification lens blank (FIG. 7A) and/or verification lens (FIG. 7B) which can include indicia, such as alignment or patient information indicia.

FIG. 7C illustrates alignment of indicia on a verification lens blank with a reticle of the laser ablation system, as seen through the system microscope.

FIGS. 7D-7E are top and side views which illustrate embodiments of a verification lens or lens blank which include pre-alignment markings

FIG. 8 schematically illustrates wavefront measurements of the eye with a wavefront sensor.

FIG. 9 illustrates a measured wavefront shape for generating an eye ablation pattern.

FIG. 10 illustrates a measured ablation of a verification lens based on the measured wavefront shape of FIG. 9.

### DESCRIPTION OF THE SPECIFIC EMBODIMENTS

Embodiments of the present invention are particularly useful for enhancing the accuracy and efficacy of laser eye surgical procedures such as photorefractive keratectomy (PRK), phototherapeutic keratectomy (PTK), laser in situ keratomileusis (LASIK), and the like. Preferably, embodiments of the invention can provide verification of the improvement of the optical system in the eye and provide feedback to physicians before the vision correction procedures. All references referred to in this application are hereby incorporated herein by reference.

The system of the present invention can be easily adapted for use with existing laser systems. By providing verification of actual improvements of the optical system in the eye, embodiments of the invention also allow the physician to evaluate the procedure plan, and whether additional measurements or an alternative plan should be prepared. Thus, the feedback provided by embodiments of the invention may facilitate sculpting of the cornea so that the eye meets and/or exceeds the normal 20/20 threshold of desired vision. In various embodiments, additional vision criteria may used either alone or in combination with an evaluation of acuity. For example, an embodiment of the invention provides patient feedback on near acuity and/or long-term effects, optionally providing near acuity of J3 or better and in some cases J1 or better.

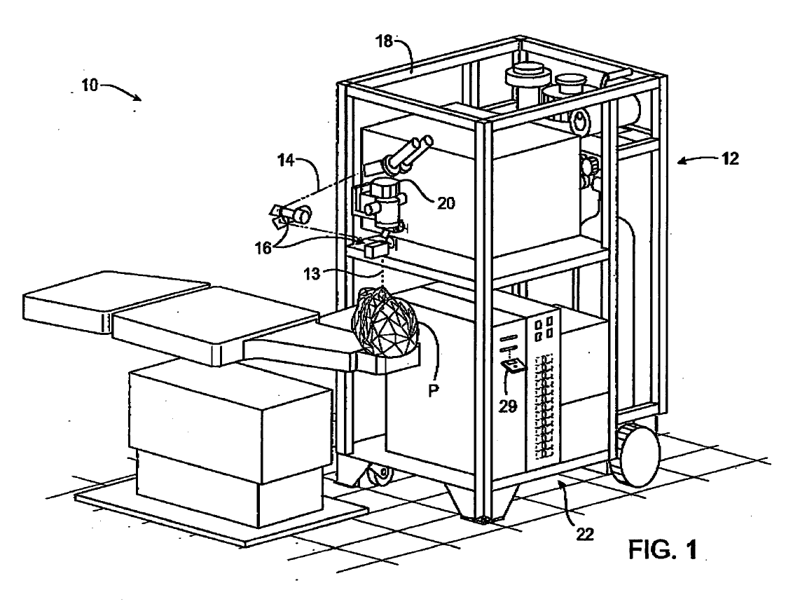

Referring now to FIG. 1, a laser eye surgery system 10 of the present invention includes a laser 12 that produces a laser beam 14. Laser 12 is optically coupled to laser delivery optics 16, which directs laser beam 14 to an eye of patient P. A delivery optics support structure (not shown here for clarity) extends from a frame 18 supporting laser 12. A microscope 20 is mounted on the delivery optics support structure, the microscope often being used to image a cornea of eye E.

In various embodiments laser 12 comprises an excimer laser, which in a preferred embodiment comprises an argon-fluorine laser configured to produce pulses of laser light having a wavelength of approximately 193 nm. Laser 12 will preferably be designed to provide a feedback stabilized fluence at the patient's eye, delivered via delivery optics 16. Embodiments of the invention may also be useful with alternative sources of ultraviolet or infrared radiation, particularly those adapted to controllably ablate the corneal tissue without causing significant damage to adjacent and/or underlying tissues of the eye. Such sources include, but are not limited to, solid state lasers and other devices which can generate energy in the ultraviolet wavelength between about 185 and 215 nm and/or those which utilize frequency-multiplying techniques. Hence, although an excimer laser is the illustrative source of an ablating beam, other lasers may be used in the present invention. Also, in other embodiments, system 10 need not be a laser based system but can be any optical or other lens profiling system known in the art for producing a verification lens such as a scleral contact lens.

Laser 12 and delivery optics 16 will generally direct laser beam 14 to the eye of patient P under the direction of a computer or processor 22. Processor 22 will generally selectively adjust laser beam 14 to expose portions of the cornea to the pulses of laser energy so as to effect a predetermined sculpting of the cornea and alter the refractive characteristics of the eye. In many embodiments, both laser 14 and the laser delivery optical system 16 will be under computer control of processor 22 to effect the desired laser sculpting process, with the processor ideally altering the ablation procedure in response to inputs from the optical feedback system described herein below. The feedback will preferably be input into processor 22 from an automated image analysis system, or may be manually input into the processor by a system operator using an input device in response to a visual inspection of analysis images provided by the optical feedback system. Processor 22 will often continue and/or terminate a sculpting treatment in response to the feedback, and may optionally also modify the planned sculpting based at least in part on the feedback.

Laser beam 14 may be adjusted to produce the desired sculpting using a variety of alternative mechanisms. The laser beam 14 may be selectively limited using one or more variable apertures. An exemplary variable aperture system having a variable iris and a variable width slit is described in U.S. Pat. No. 5,713,892, the full disclosure of which is incorporated herein by reference. The laser beam may also be tailored by varying the size and offset of the laser spot from an axis of the eye, as described in U.S. Pat. Nos. 5,683,379 and 6,203,539 and 6,331,177 the full disclosures of which are incorporated herein by reference.

Still further alternatives are possible, including scanning of the laser beam over the surface of the eye and controlling the number of pulses and/or dwell time at each location, as described, for example, by U.S. Pat. No. 4,665,913 (the full disclosure of which is incorporated herein by reference) and as demonstrated by other scanning laser systems such as those manufactured by LaserSight, Alcon/Summit/Autonomous, WaveLight Technologies AG, Chiron Technolas, and by Bausch and Lomb. Other approaches can include using masks in the optical path of laser beam 14 to vary the profile of the beam incident on the cornea and using hybrid profile-scanning systems in which a variable size beam (typically controlled by a variable width slit and/or variable diameter iris diaphragm) is scanned across the cornea as described in U.S. Pat. No. 6,673,062, the full disclosure of which is incorporated herein by reference. The computer programs and control methodology for these laser pattern tailoring techniques are well described in the patent literature.

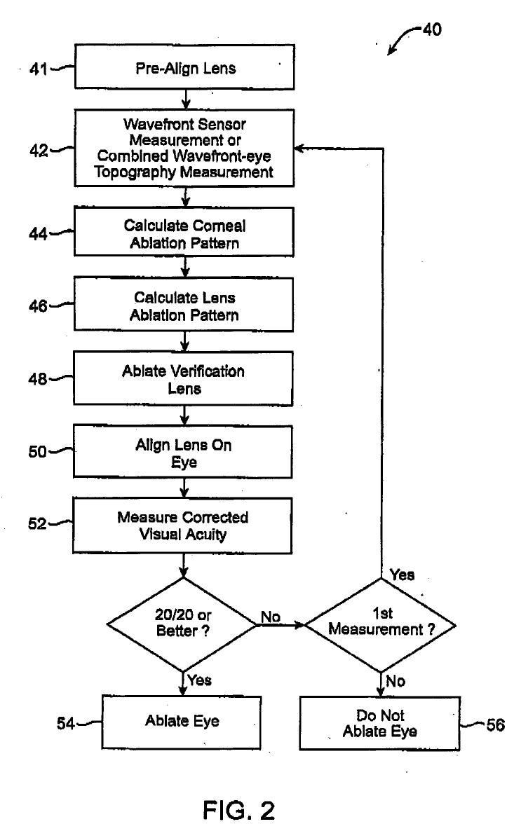

Referring now to FIGS. 2-5, an exemplary verification and refraction correction method 40 will now be discussed. The order of steps shown is exemplary and other orders and/or additional or fewer steps may be used. Various embodiments of method 40 can be used to verify that an intended ablation, (e.g., a laser ablation) is appropriate for a particular eye. Measurements of the eye are taken, ideally to determine both standard refractive errors (e.g., myopia, hyperopia, and/or astigmatism) and irregular refractive errors (optionally including any other optical errors of the optical system of the eye). In the exemplary method, the optical errors of the eye are measured in a measurement step 42 with a wavefront sensor system 60, such as the WaveScan® system available commercially from VISX, Incorporated, the system described in U.S. Pat. No. 6,095,651, or the like. However, other instruments and methods for measurement of optical error may also be used.

Based on the measurements of the eye, a corneal ablation pattern may be calculated 44 by processor 22 (or by a separate processor) for ablating the eye with system 10 so as to correct the optical errors of the eye. Such calculations will often be based on both the measured optical properties of the eye and on the characteristics of the corneal tissue targeted for ablation (e.g., the ablation rate, the refractive index, the propensity of the tissue to form “central islands” or decreased central ablation depths within a uniform energy beam, and the like). The results of the calculation will often comprise an ablation pattern in the form of an ablation table listing ablation locations, numbers of pulses, ablation sizes, and or ablation shapes to effect the desired refractive correction. The ablation table in turn can be stored in an electronic database known in the art (e.g., a relational database) and/or in memory resources known in the art (e.g., RAM, ROM, etc.). An exemplary method for generating ablation patterns is described in U.S. Pat. No. 6,673,062, the full disclosure of which is incorporated herein by reference.

Rather than directly proceeding to the ablation, another ablation pattern may also be calculated 46 for ablation of a verification lens 90. In an embodiment, verification lens 90 can be a scleral contact lens 90s having a peripheral portion 90pp and a central portion 90cp as is described herein. The ablation pattern for the verification lens may be calculated based on the measured optical properties of the eye, together with the characteristics of a lens material including the refractive index of the lens material, the ablation rate of the lens material, any ablation shape-effects of the lens material, and/or the like. The verification lens may then be aligned with the ablation system and ablated 48 using system 10 or optionally, using a system similar to that shown in U.S. Pat. No. 6,638,271 which is also incorporated herein by reference. However, other contact lens laser ablation systems known in the art may also be used. For embodiments using a scleral lens, the ablation pattern is imposed on the central portion of the lens so that the resulting optical profile of the central portion corresponds with the proposed ablative (or other) correction of the eye.

After the verification lens has been generated, the lens is positioned on the patient's eye and evaluated for proper fit and/or alignment 50. Alignment step 50 can be accomplished using visual observation and/or other contact lens fitting methods known in the art. In one embodiment, the doctor can verify that the central portion 90cp is aligned with the cornea C using topographic measurement system 100 or other eye measurement means known in the art. System 100 can also be used to verify that the curvature of lens profile 90p matches that of corneal profile Cp. In embodiments where lens 90s has alignment indicia 92a (described herein), alignment step 50 can be facilitated by a visual determination to make sure that the alignment indicia 92a align with iris patterns IP, the Limbus Li or other feature of the eye. If the lens is not properly aligned, the physician can perform an in situ alignment manually or with the aid of a corneal keratometer or topographic measurement system 100 or other corneal/contact lens instrument known in the art. Proper alignment provides a higher correlation between the corrective effect produced by the verification lens and the corrective effect of the intend eye ablation procedure.

In various embodiments, a pre-alignment step 41, may be done prior to wavefront measurements. Similar to alignment step 50, pre-alignment can be accomplished using visual observation and/or other contact lens fitting methods known in the art. Pre-alignment may be particularly useful where the wavefront is subsequently measured with the scleral lens in place, using registration marks (described herein) indicating how the scleral lens rests on the cornea. Embodiments having pre-alignment provide a means for improving the correlation between the correction produced by the verification lens and the subsequent ablation procedure. In alternative embodiments, a combined wavefront-topography system can be used to make measurements which account for aberrations of the eye as well as the surface contour of the eye for fitting the lens.

After an alignment/fitting determination, visual performance using the verification lens can be assessed 52. Visual performance assessment 52 can be done immediately after the fitting determination, that same day after the patient has worn the lens for a number of hours or even after the patient has worn the lens for a number of days (e.g., two or more) though not necessarily continuously. The types of visual determination which can be made include without limitation, measurement of visual acuity (e.g., using a standard eye chart), depth of field, accommodation, contrast sensitivity and combinations thereof. One or more of these tests may be done under varying lighting conditions. The patient could also complete a subjective visual performance questionnaire. Information from one or more of these tests could be stored on a database and be used for evaluation of subsequent visual corrective plans for the particular patient, or a patient population or even a sub-population (e.g., pediatric patients or myopic patients). When using a scleral verification lens 90s, prior to the visual assessment, the patient may register the verification lens with their eye by positioning the peripheral portion of the lens on the sclera so that the central portion of the lens is optically aligned with aberrations of the eye.

Visual performance of the verification lens may be assessed by having the patient scan an eye chart to determine visual acuity. If the measured visual acuity is equal to or better than some predetermined threshold value, often 20/20 or better and optionally 20/15 or better, the eye is ablated with the planned ablation pattern 54. If not, a second measurement may be taken and the process repeated, and if acuity still remains unacceptable, the ablation may not be performed 56.

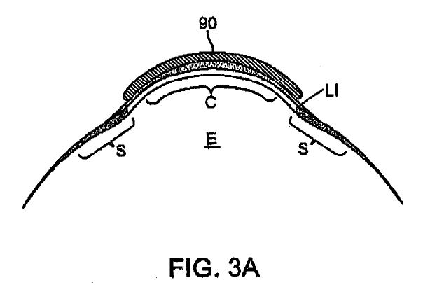

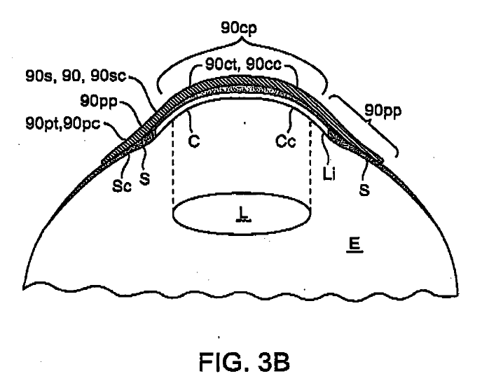

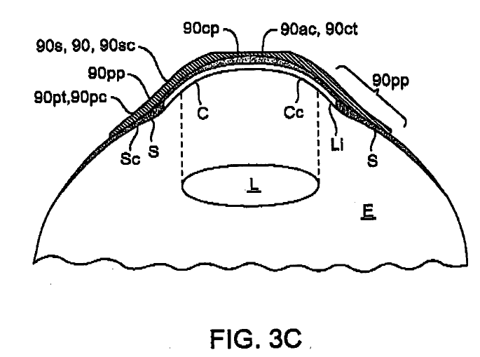

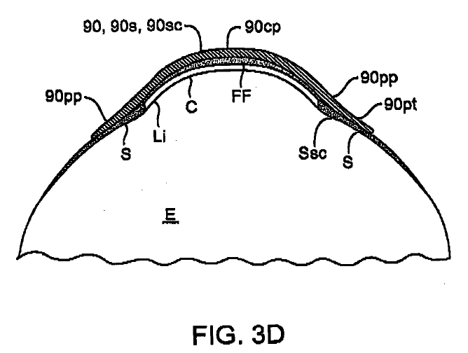

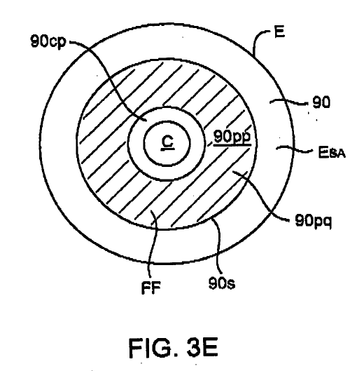

Referring now to FIGS. 3A-E, a discussion will now be presented of verification lens 90. Current contact lenses include corneal lenses and scleral lenses. For purposes of this disclosure, verification lens 90 is a scleral lens 90s. As shown in FIG. 3A, corneal lenses are primarily positioned over the cornea C of the eye E. However, because of the smaller surface area of the corneal lens in relation to the surface area of the eye, corneal lenses are prone to movement over the eye from a number of factors including movement of the head, movement of the eye or even blinking. Such movement may impair or impede the patient's ability to evaluate the optical performance of the lens through one or more evaluation described herein. In particular, the movement may result in misalignment of the lens with the higher order aberrations of the eye sought to be corrected.

A scleral contact lens may overcome the stability limitations of a corneal lens. As shown in FIG. 3B, a scleral lens 90s, extends over the cornea C and onto the sclera S, the white outer coating of the eye E which surrounds the cornea. Scleral lens 90s can be configured to cover all of the cornea and selectable portions of the sclera. Lens 90s includes two portions, a central portion 90cp which covers the cornea and a peripheral portion 90pp which covers a selectable portion of the sclera S. The central portion 90cp has a corrective optical profile corresponding to a calculated corneal ablation pattern or that from another proposed vision correction treatment. The central portion 90cp is adapted to be optically aligned with the lens L of eye E, i.e., portion 90cp extends substantially over the entire portion of the cornea C overlying the lens L. Also, central portion 90cp can include a contour 90ct having a curvature 90cc corresponding to Corneal curvature Cc and peripheral portion 90p can include a contour 90pt having a curvature 90pc corresponding to scleral curvature Sc. The contour 90ct of central portion 90cp can be configured such that central portion 90cp does not directly contact cornea C. Further description of scleral contact lenses is found in U.S. Pat. No. 5,929,968 which is incorporated by reference herein.

In many embodiments, the scleral contact 90s can not only be configured as a verification lens, but also as a corrective lens 90sc which can be worn by the patient to correct their vision as other corrective contact lens are used (e.g., hard and soft contact lens, etc). In various embodiments, lens 90sc can be configured as a daily wear lens, or an extended wear lens. In preferred embodiments, lens 90sc is fabricated using wavefront-driven measurements of aberrations of the eye, including measurements of irregular aberrations, as is described herein. Such methods can also incorporate measurement of the topography of the eye using, e.g., a corneal topographic measurement system described herein. These measurements can be done before or after wavefront measurements. After the corrective lens is fabricated, the wavefront measurement can also be repeated with the corrective lens in place on the eye, to verify the correction of the lens. The information from wavefront measurements with the lens in the eye, can also be used to titrate or fine tune the corrective profile of the corrective lens using lens fabrication methods described herein (e.g., lens ablation methods). Corrective lens 90sc can be a soft or hard contact lens and can thus be fabricated using soft or hard contact lens materials and processing methods known in the art including gas permeable materials and technology. Also, as is described below, corrective lens 90sc can be configured to have an aspheric shape to correct for standard errors, such as refractive errors, as well as irregular errors such as higher order aberrations and presbyopia.

In various embodiments, the scleral lens can be configured to have an aspheric shape or contour 90ac as is shown in FIG. 3C. Aspheric contour 90ac can have an optical profile configured to correspond to an ablation pattern or other proposed correction to treat various conditions of the eye such as refractive errors, higher order aberrations and presbyopia. The higher order aberrations can include, without limitation, 2nd, 3rd, 4th or even higher order aberrations as determined by Zernike analysis and/or other wavefront analysis methods described herein. The aspheric contour can be configured to correspond to an optical profile for one or all of these conditions or other aberrations of the eye. Thus embodiments having an aspheric shaped scleral lens can be used to verify proposed treatments and/or to correct for a combination of optical errors including standard errors, such as refractive errors, and irregular errors such as higher order aberrations and presbyopia.

The peripheral portion 90pp of the lens can extend radially from over the outer portions of the cornea then extend over the limbus Li and then over selectable portions of the sclera S. Alternatively, the central portion 90cp can extend over the entire cornea C (even into the limbus Li), with the peripheral portion 90pp beginning in the limbus Li. In many embodiments, the peripheral portion can extend sufficiently over the sclera such that it underlies the eye lid even when the eye is open. This provides one means for stabilizing lens 90s on the eye and reducing movement of the lens from blinking, head movement etc. Other means are discussed below.

The peripheral portion 90pp can be configured to stabilize lens 90s on the eye so as to reduce lens movement from blinking, eye movement, head movement and other biomechanical movements. In an embodiment shown in FIG. 3D, this can be accomplished by configuring the peripheral portion 90pp to have a contour 90pt corresponding to a surface contour of the sclera Ssc so that the peripheral contour 90pt stabilizes the verification lens on the eye. In a related embodiment shown in FIG. 3E, the peripheral portion 90pp can be also configured to have sufficient surface area 90pa in relation to the exposed surface area Esa of the open eye (i.e., approximately the hemispherical area of the eye), such that lens 90s is held in place by the adhesive forces between the lens and the fluid film FF which sits above the surface of the eye. That is, these adhesive forces are sufficient to overcome inertial forces from head, neck and eye movement or any mechanical forces imparted by blinking. The size of the peripheral portion 90pp (and/or lens 90s) can be selected such the ratio of the surface area 90pa of the peripheral portion to that of the exposed eye surface area Esa can be in the range of about 4:1 to about 1:1, with specific embodiments of 3:1 and 2:1. In related embodiments, the mechanical properties (e.g., flexibility) of the scleral lens can be configured to substantially match those of underlying portions of the eye (e.g., the sclera) such that the two substantially behave as one mechanical body. In one embodiment, this can be achieved by configuring the stiffness profile of lens 90s to substantially match the stiffness profile of the underlying section of the eye.

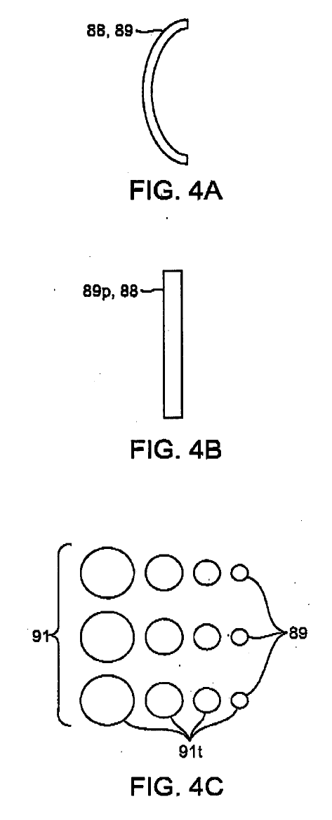

A discussion will now be presented of fabrication methods for a scleral lens. In various embodiments, lens 90s can be fabricated using laser ablation methods described herein or known in the art as well conventional contact lens fabrication methods. Referring now to FIGS. 4A-4C, lens 90s can be fabricated from a lens workpiece 88. Typically, workpiece 88 is a contact lens blank 89 comprising contact lens material 89m. In one embodiment, lens blank 89 can be a plano lens 89p, with the desired amount of lens curvature produced through the laser ablation process (note, plano lens 89p need not be flat but, can have any selectable profile). Alternatively, lens blank 89 can be selected from the lens fitting set 91, or another lens having a curvature and/or size corresponding to either a lens selected from the lens fitting set or to topographic measurements of the eye. Further, in various embodiments, lens blank 89 can be selected to have various amounts of pre-ablation curvature and/or optical correction. The amount of pre-ablation curvature and/or correction can be based on initial measurements of the patient's eye and can allow for a faster and more accurate lens ablation process.

Suitable lens materials for lens blank 89 can include a variety of contact lens materials including a PMMA (polymethylmethacrylate) fluorocarbon copolymer, silicon acrylate and combinations thereof or other gas permeable lens materials known in the art. Also, the lens material can be selected to produce a rigid or flexible lens as well as a gas permeable lens and can thus include any number of gas permeable polymers known in the art. In a preferred embodiment, the lens material is a gas permeable lens material.

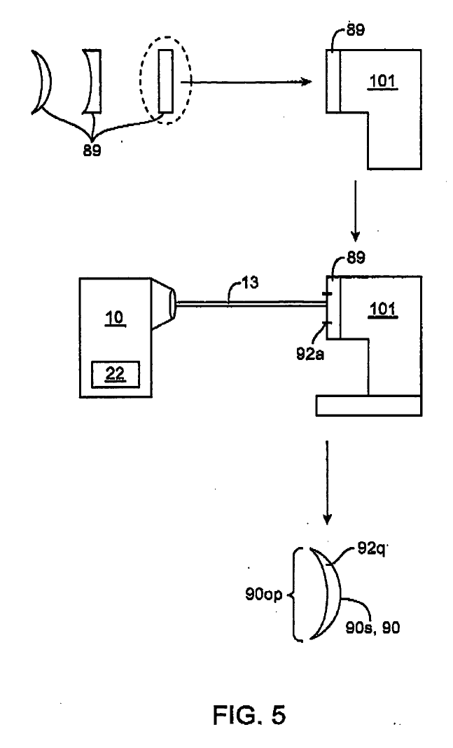

FIG. 5 illustrates an exemplary method 120 for fabricating a verification contact lens 90 such as a scleral lens 90s. A desired contact lens blank 89 is selected and then aligned in the focal path 13 of the laser system 10. Alignment can be facilitated by placing the lens blank in an alignment fixture 101 or through the use of indicia 92 on the lens blank, see discussion below. The lens blank 89 is then ablated using system 10 according to an ablation pattern corresponding to an ablation pattern for the eye to produce a desired corrective optical profile 900p. One or both surfaces of the lens blank can be ablated. Also, for embodiments using a curved lens blank, either the concave or convex surface of the lens can be ablated. In addition to ablating the lens blank to produce a desired optical profile, system 10 can also be used to deliver laser energy to create one or more indicia 92, including alignment indicia 92a, on lens 90s. Alignment indicia 92a can be configured to align with one or more features on the eye, to facilitate proper alignment of the lens on the eye. In one embodiment, the indicia can be configured to align with iris patterns IP describe herein.

All or a portion of the ablation process can be controlled using processor 22 or other electronic control means known in the art. Accordingly processor 22 can be configured to calculate, modify or store the desired ablation pattern. Also processor 22 can be configured to control the generation of the alignment indicia 92a. In one embodiment, a stream of inert gas such as nitrogen can be blown over the lens before, during or after ablation. Other suitable gases include argon and helium. The flow of the gas can be controlled by processor 22 to increase or decrease the flow rate and/or velocity as needed. In various embodiments, processor 22 or other control means can be used to control one or more gas flow parameters responsive to one or more of the temperature of blank 89/lens 90s, rate of ablation, optical fluence, laser intensity, laser power level and the like. In various embodiments, ablation of the verification lens 90 can be performed in a vacuum or pressure chamber not shown.

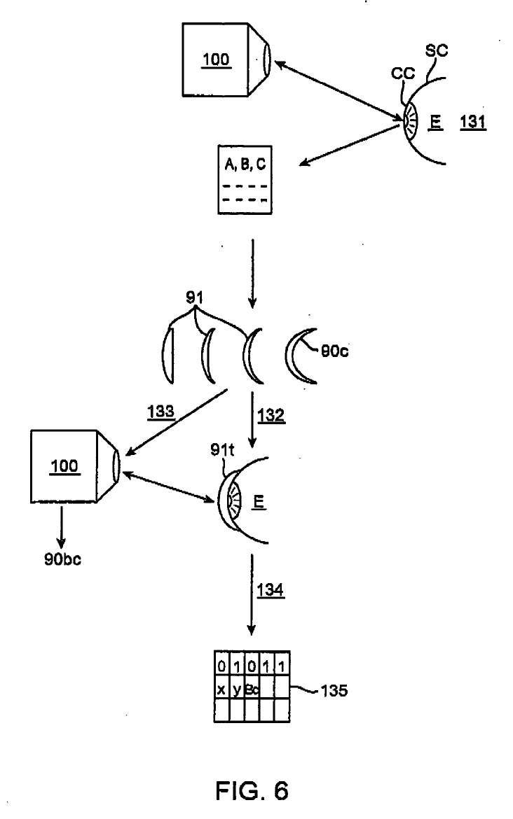

Referring now to FIG. 6, a discussion will now be presented of an exemplary method 130 for fitting a verification lens 90, such as a scleral lens 90s, onto the eye. First ocular health evaluations, corneal shape measurements, static or dynamic pupil information (e.g., pupil size for different lighting conditions and focus points) and other ocular biometric data 131 can be taken in an information gathering step 132. This information is used in combination with a sclera lens fitting set 91 to facilitate selection and fit of the scleral lens. Various trials lenses 91t from the fitting set are tried on the patient in a fitting step 132 and used to select a lens having the proper base curvature 90c. Determination of base curvature 90c can include or otherwise be facilitated by a topographic measurement such as measurement of corneal curvature Cc and/or scleral curvature Sc which can be done as part of information gathering step 131 or as a separate measurement 133. Measurement step 133 can be done before or after fitting step 132. These and related eye topographic measurements can be made using an eye topographic measurement system 100 and related methods known in the art. In various embodiments, system 100 can include a corneal topographer or corneal keratometer known in the art. Base curvature can also be determined/confirmed by evaluation of one or more of lens stability, rotation and alignment with parts of the eye (e.g., with the cornea). Determination of the base curvature together with other ocular biometric data (e.g., pupil data (e.g., size, dynamics etc.) and eye aberrations (e.g., refractive error and higher order aberrations)) can be used to generate 134 an ablative treatment data set 135 for fabricating a scleral fabrication lens 90s. In an embodiment, data set 135 can be stored, accessed or manipulated using an electronic database known in the art such as a relational database.

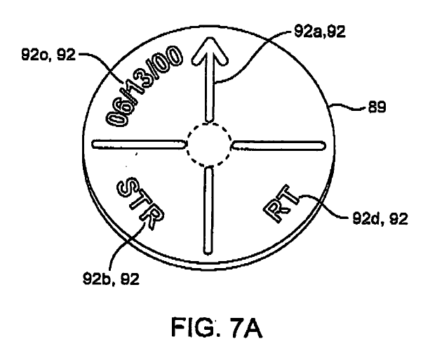

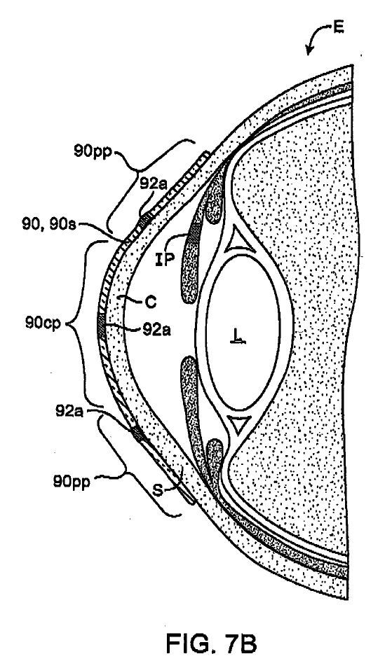

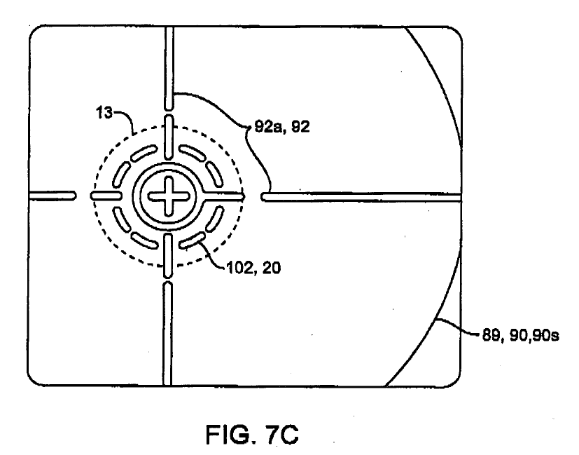

Referring now to FIGS. 7A-7E, a discussion will now be presented of embodiments of lens blank 89 and/or verification lens 90 which can include indicia 92 or other features or markings. The indicia can created before, during or after the ablation procedure. The indicia can be generated before or after ablation using contact lens imprinting an/or fabrication methods known in the art or during lens ablation using system 10. In various embodiments, the indicia can provide an indication of lens orientation, position and other information. In one embodiment, the indicia can include alignment indicia 92a used to approximately align lens blank 89 in the focal path of laser system 10. Alignment indicia 92a can be placed in locations corresponding to the peripheral portions 90pp and central portions 90cp of a scleral verification lens 90s. In another embodiment discussed above, the indicia can include alignment indicia 92a used to align verification lens 90 on the eye prior to evaluation of the verification lens. The indicia may also indicate an identity of the patient 92b, a date of a procedure and/or measurement 92c, a particular eye (left or right) of the patient 92d, a doctor, a system or treatment tracking number, or the like as is shown in FIG. 7A. In a preferred embodiment, the lens includes three alignment indicia 92a which are positioned at the top (i.e., superiorly with respect to the head of the wearer) and right and left portions of the lens.

As shown in FIG. 7C, alignment indicia 92a can be alignable with a reticle 102 of a microscope or other optical instrument 20 (e.g., a video camera) of system 10. This in turn allows a more precise alignment of verification lens blank 89 with respect to selected optical reference positions of system 10, e.g., with respect to the focal path 13 of system 10. The aligned lens blank 89 may then be ablated as described above using the laser ablation system shown in FIG. 1 or other ablation system described herein. In one embodiment, the verification lens blank 89 is supported at the location typically occupied by the cornea of the patient. By producing more precise lens alignment, alignment indicia 92a allow for the generation of a verification lens having a refractive correction more closely correlated to that of an intended eye corrective treatment, such as an ablative treatment. This in turn, allows for a more accurate and reliable verification of an intended eye corrective treatment.

In various embodiments, lens blank 89 or the scleral lens 90s may have pre-registration marks or features 92p that will facilitate alignment of the lens on the eye. As shown in FIGS. 7D-7E, these marking can be positioned at various locations on lens blank 89 or lens 90s. Lens blank 89 can include various numbers of pre-registration marks 92p. In preferred embodiments, the lens blank includes between one to ten marks 92p. In the embodiment shown in FIG. 7D-7E the lens blanks includes three pre-registrations marks 92p. Also, the marking can be radially distributed (including a substantially equal radial distribution) with respect to a circumference 89C of the lens blank.

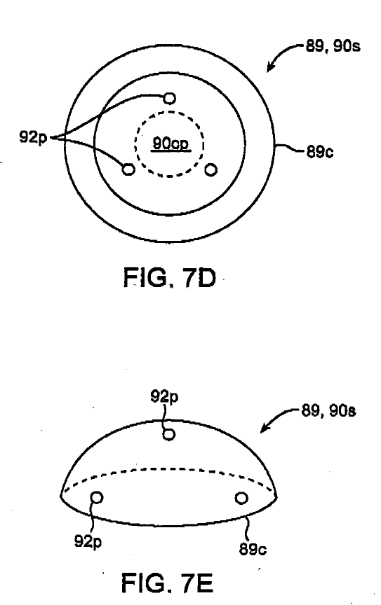

Referring now to FIGS. 8-10, a discussion will now be presented of wavefront measurement systems and methods that can be used in various embodiments of the invention. FIG. 8 schematically illustrates wavefront measurements of the eye with a wavefront sensor system 60, as generally described above. Wavefront system 60 projects light 62 using optics 64 toward eye E. Light 62 is transmitted by cornea C of eye E, and forms a retinal image 66 on the retina R of eye E. Wavefront sensor 60 typically forms a series of images 68a, 68b, 68c, (collectively images 68) on a sensor surface 70, often using a microlens array 72 in combination with at least a portion of optics 64. Images 68, 68b, 68c, are each also formed in part by a corresponding portion of cornea C, so that the images 68 can be analyzed to determine local refractive properties and errors across the cornea. These wavefront analysis techniques optionally make use of Zernike polynomials. Alternative analysis methods and wavefront systems are described in U.S. patent application Ser. Nos. 10/006,992, 10/601,048, and 10/872,107, the full disclosure of each of which is incorporated herein by reference.

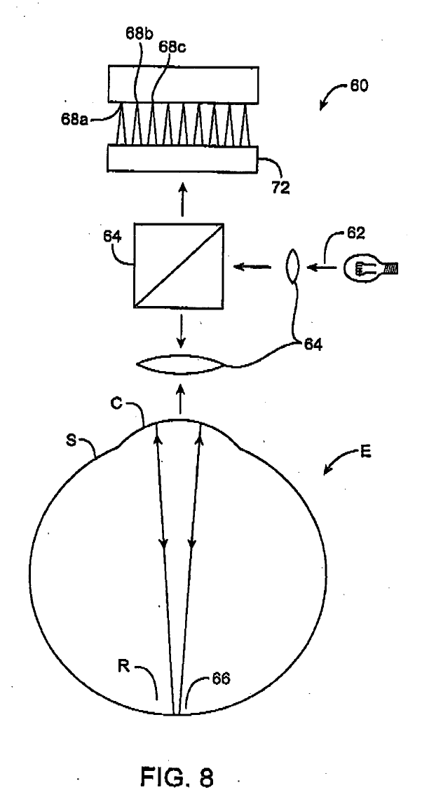

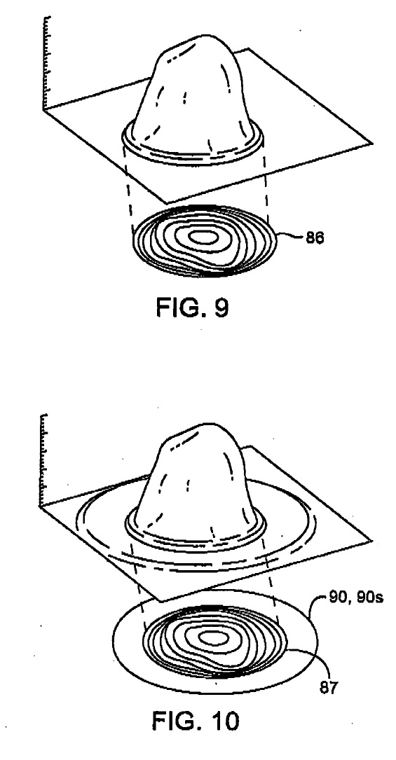

As shown in FIG. 9, system 60 can be used to produce a measured wavefront shape 86. As shown in FIG. 10, measured wavefront shape 86 can, in turn, be used to generate an ablation pattern 87 of a verification lens 90, such as scleral lens 90s that is used to verify an intended ablative or other eye treatment to correct aberrations of the eye. Such embodiments can be used in a method for verifying the vision correction of they eye wherein the correction process and/or the verification is wavefront-driven.

The foregoing description of various embodiments of the invention has been presented for purposes of illustration and description. It is not intended to limit the invention to the precise forms disclosed. Many modifications, variations and refinements will be apparent to practitioners skilled in the art. Further, elements or acts from one embodiment can be readily recombined with one or more elements or acts from other embodiments. Also, elements or acts from one embodiment can be readily substituted with elements or acts of another embodiment. Hence, the scope of the present invention is not limited to the specifics of the exemplary embodiment, but is instead limited solely by the appended claims.

What is claimed is:

1. A method for verifying vision correction for an eye of a patient, the eye having a cornea surrounded by a sclera, the method comprising:
measuring irregular aberrations of the eye;
determining a proposed refractive correction for treatment of the aberrations;
configuring a central portion of a verification lens so that the central portion corresponds with the proposed correction;
positioning a peripheral portion of the verification lens upon the sclera so that the central portion is optically aligned with the aberrations; and
determining whether a corrected vision of the eye with the verification lens is acceptable so as to verify the proposed correction.

2. The method of claim 1, wherein the peripheral portion stabilizes the verification lens on the eye during the determination of vision acceptability.

3. The method of claim 1, wherein the peripheral portion has a surface contour corresponding to a surface contour of the sclera so that the peripheral contour stabilizes the verification lens on the eye.

4. The method of claim 1, wherein the verification lens has an aspheric shape configured to correspond to a correction for higher order aberrations of the eye.

5. The method of claim 1, wherein the lens has an aspheric shape configured to correct for presbyopia.

6. The method of claim 1, wherein the lens is determined to be acceptable by evaluation of a property selected from the group consisting of a wavefront of the eye with the verification lens thereon, visual acuity, accommodation or contrast sensitivity.

7. The method of claim 1, wherein the determination is completed after the patient has worn the verification lens for a plurality of hours.

8. The method of claim 1, wherein the aberrations are measured by measuring a wavefront of light passed through the optical components of the eye while the lens is on the eye.

9. The method of claim 8, wherein the verification lens includes indicia of alignment, the method further comprising altering refractive properties of the verification lens per the wavefront with reference to the indicia of alignment

10. The method of claim 9, wherein the acceptability of the corrected vision is determined by verifying a position of the verification lens on the eye using the indicia of alignment.

11. The method of claim 1, wherein the central portion is configured by ablating the central portion with a profile corresponding to the proposed correction.

12. The method of claim 11, wherein the ablation profile is imposed on the anterior surface by a laser ablation system, the method further comprising laser ablating the eye with the laser ablation system according to the proposed refractive correction.

13. The method of claim 1, further comprising:
generating a surgical profile to treat the eye, the profile being modified from the proposed correction in response to the determination of acceptability.

14. The method of claim 1, wherein the refractive correction mitigates at least one of higher order optical aberrations, lower order optical aberrations or presbyopia.

15. A method for correcting the vision of an eye of a patient, the eye having a cornea surrounded by a sclera, the method comprising:
measuring irregular aberrations of the eye;
determining a refractive correction for the aberrations;
configuring a central portion of a lens so that the central portion corresponds with the proposed correction;
positioning a peripheral portion of the lens upon the sclera so that the central portion is optically aligned with the aberrations; and
utilizing the lens to correct for the aberrations of the eye of the patient.

16. The method of claim 15, wherein the correction includes a correction for presbyopia.

17. The method of claim 15, wherein the lens has an aspheric shape configured to correct for at least one of a higher order condition or presbyopia.

18. The method of claim 15, wherein the aberrations are measured by measuring a wavefront of light passed through the optical components of the eye while the lens is on the eye.

19. The method of claim 18, wherein the measurement of the aberrations of the eye includes measurement of the topography of the eye.

20. The method of claim 18, wherein the lens includes indicia of alignment, the method further comprising:
registering the correction with the lens using the indicia of alignment, and
registering the configured lens with the eye using the indicia of alignment.

21. A system for correcting irregular aberrations of an eye of a patient, the eye having a cornea surrounded by a sclera, the system comprising:
a sensor for measuring the irregular aberrations of the eye;
a processor for generating a refractive prescription using the measured aberrations;
a lens workpiece having a central portion and a peripheral portion, the peripheral portion configured to be positioned on the sclera; and
a lens configuring system for forming an aberration correcting lens from the lens workpiece per the refractive prescription such that the aberrations are mitigated.

22. The system of claim 21, wherein the processor generates a verification pattern of laser energy corresponding to the refractive prescription.

23. The system of claim 21, wherein the lens configuring system is a laser system for directing laser energy onto the lens workpiece according to the refractive prescription so as to form the aberration correcting lens.

24. The system of claim 23, wherein the lens workpiece includes indicia of alignment, the system further comprising an alignment system configured to align an ablative beam from the laser system with the lens workpiece utilizing the indicia of alignment.

25. The system of claim 21, wherein laser energy is directed to impose the verification pattern on the central portion of the lens workpiece.

26. The system of claim 21, wherein the peripheral portion is configured to optically align the central portion with aberrations on the eye.

27. The system of claim 21, wherein the peripheral portion is configured to stabilize the verification lens on the eye.

28. The system of claim 21, wherein the peripheral portion is configured to support the central portion over the eye.

29. The system of claim 21, wherein the sensor is configured to measure a wavefront of light passed through the optical components of the eye and the lens workpiece.

30. A system for correcting an irregular aberration of an eye, the system comprising:
a lens workpiece having a central portion and a peripheral portion, the peripheral portion configured to be positioned on the sclera, the workpiece having indicia of alignment;
a wavefront sensor configured to measure a wavefront from the eye with the lens workpiece thereon such that the wavefront can be referenced to the indicia of alignment;
a processor configured to determine changes to the lens workpiece, the processor having an output transmitting refractive alterations for the lens workpiece referenced to the indicia of alignment; and
a laser system coupled to the output of the processor, the laser system configured to generate an ablative beam for implementing the refractive alterations with reference to the indicia of alignment.

31. A scleral lens for correcting an aberration of an eye of a patient, the lens comprising a central portion and a peripheral portion, the peripheral portion configured to be positioned on the sclera and stabilize the lens on the eye, the central portion having an optical profile corresponding to a refractive correction for the aberration wherein the optical profile is determined using a wavefront-driven measurement of the aberration.

32. The lens of claim 31, wherein the optical profile is created by laser ablation.

33. The lens of claim 31, the refractive correction mitigates at least one of higher order optical aberrations, lower order optical aberrations or presbyopia.

34. The lens of claim 31, wherein the peripheral portion has a surface contour corresponding to a surface contour of the sclera so that the peripheral contour stabilizes the lens on the eye.

35. The lens of claim 31, wherein the lens has an aspheric contour.

36. A scleral lens for correcting an aberration of an eye of a patient, the lens comprising a central portion and a peripheral portion, the peripheral portion configured to be positioned on the sclera and stabilize the lens on the eye, the central portion having an optical profile corresponding to a refractive correction for the aberration, the optical profile determined by a wavefront-driven measurement of the aberration and produced by laser ablation of a surface of the lens.
